(19) United States
(12) Patent Application Publication
(10) Pub. No.: US 2012/0130207 A1
(43) Pub. Date: May 24, 2012

(54) MICRO-NEEDLE DEVICE AND APPARATUS AND A METHOD FOR APPLYING A MICRO-NEEDLE ELEMENT TO A SITE ON THE SKIN OF A SUBJECT

(75) Inventors: John O'dea, Bearna (IE); Eoin Bambury, Navan (IE)

(73) Assignee: JANISYS LIMITED, Dangan, Galway (IE)

(21) Appl. No.: 13/266,248

(22) PCT Filed: Apr. 29, 2010
(86) PCT No.: PCT/IE10/00032

§ 371 Date: Jan. 20, 2012

(30) Foreign Application Priority Data
Apr. 29, 2009 (IE) S2009/0337

(51) Int. Cl. A61B 5/053; A61B 5/145; A61M 5/00; A61B 5/103

(52) U.S. Cl. 600/309; 600/557; 600/547; 604/506; 604/503

(57) ABSTRACT

A micro-needle device (3) comprises a micro-needle element (2) which is carried on a strap (14) and is securable around a limb of a subject by the strap (14) with the micro-needle element (2) located at a desired site. A pressure sensor (23) is located between the strap (14) and the micro-needle element (2) for detecting the pressure at which the micro-needle element (2) is urged into engagement with the skin of the subject. An adjustable clasp (18) secures the strap (14) and permits adjustment of the tension in the strap (14) for in turn adjusting the pressure with which the micro-needle element (2) is urged into engagement with the skin of the subject. An electronic control circuit located in a housing (26) mounted on the strap (14) monitors the pressure read by the pressure sensor (23) and operates three light emitting diodes (28,29,30) to indicate the pressures with which the micro-needle element (2) is urged into engagement with the skin of the subject, and also to indicate when the micro-needle element (2) is urged into engagement with the skin of the subject by the strap (14) with micro-needles (5) of the micro-needle element (2) penetrating the skin of the subject to a desired depth.

The present invention relates to apparatus and a method for applying a micro-needle element to a site on the skin of a subject, and the invention also relates to a micro-needle device.

Micro-needle devices for administering a medicament to a subject are known. Such micro-needle devices typically comprise a patch type device, which includes a micro-needle element comprising a housing and a plurality of micro-needles, typically of the order of 0.2 mm in length extending from the housing. A plurality of reservoirs are located in the housing of the micro-needle element for storing one or more medicaments to be administered to the subject. In general, each micro-needle communicates with a corresponding reservoir in the housing so that the number of micro-needles and the number of reservoirs for the medicament or medicaments are similar. Discharge means are provided in the housing for discharging the medicament from each reservoir, and in general, a separate discharge means is provided for each reservoir, so that the medicaments in the respective reservoirs may be independently discharged through the corresponding micro-needles. Such patch type micro-needle devices will be well known to those skilled in the art. Typical patch type micro-needle devices are disclosed in PCT Published Application Specifications Nos. WO 2006/060106 and WO 2008/012788.

Typically, the micro-needle element is secured to a site on the skin of a subject by a self-adhesive patch, or by an adjustable strap. In general, where such a micro-needle element is to be secured to an arm of a subject, the element is attached to the arm by a strap which encircles the arm of the subject, and is secured by an adjustable clasp. On the other hand, where the micro-needle element is to be secured to a site on the skin of a subject, for example, on the abdomen or the thigh, in general, the micro-needle element is secured by a self-adhesive patch which extends over the micro-needle element and externally around and outwardly of the periphery of the micro-needle element for adhering to the skin of the subject.

However, due to the relatively short length of the micro-needles, it is essential that when a micro-needle element is being secured to the skin of the subject that sufficient pressure is applied to the micro-needle element to ensure that the micro-needles penetrate through the skin of the subject to a desired depth. Unfortunately, there is no known accurate means for determining when the micro-needles of a micro-needle element have penetrated below the skin to the desired depth, and in general, attaching such a micro-needle element to the skin of a subject tends to be very much a trial and error exercise.

Additionally, such micro-needle elements, in general, are programmable so that the discharge means for discharging the medicament or medicaments from the reservoir may be programmed to operate at appropriate times and at appropriate intervals. Additionally, such micro-needle elements also include means for recording the dates and times at which respective doses of one or more of the medicaments are discharged to a subject. This recorded data is particularly useful to a doctor, surgeon or the like for monitoring the compliance of a patient with a treatment regime. However, some patients tend to be rather careless in their use of such micro-needle elements, and the micro-needle element may be removed from the site, and reapplying the element to the site may be inadvertently omitted. During periods while such a micro-needle element has been removed from the subject, the programme of the micro-needle element continues to run, thus discharging the one or more of the medicaments at the programmed times and at the programmed intervals. Such discharging of the medicament or medicaments is recorded as though the medicament or medicaments were actually discharged into the subject, thus providing an erroneous compliance record for the micro-needle device.

There is therefore a need for a micro-needle device which addresses this problem, and there is also a need for apparatus and a method for applying a micro-needle element to a site on the skin of a subject which addresses some of the above stated problems of micro-needle elements and micro-needle patch type devices known heretofore.

The present invention is directed towards providing a micro-needle device, and the invention is also directed towards providing apparatus and a method for applying a micro-needle element to a site on the skin of a subject which addresses at least some of the problems of micro-needle elements and devices known heretofore discussed above.

According to the invention there is provided apparatus for applying a micro-needle element, which comprises at least one micro-needle, to a site on the skin of a subject, the apparatus comprising a means for urging the micro-needle element into engagement with the skin of the subject with the at least one micro-needle thereof penetrating the skin, a means for determining when the at least one micro-needle of the micro-needle element penetrates the skin to a desired depth, and an indicating means responsive to the determining means determining that the at least one micro-needle of the micro-needle element has penetrated the skin to the desired depth for producing a first human sensory perceptible signal.

In one embodiment of the invention the determining means for determining when the at least one micro-needle penetrates the skin of the subject to a desired depth comprises a pair of measuring electrodes, the measuring electrodes being urgeable by the urging means into penetrating engagement with the skin of the subject with the at least one micro-needle, and an electrical impedance detecting means for detecting electrical impedance beneath the skin of the subject between the measuring electrodes. Preferably, the measuring electrodes are located spaced apart from each other. Advantageously, the measuring electrodes are located so that the depth to which the measuring electrodes penetrate beneath the skin of the subject in response to the urging means urging the micro-needle element into engagement with the skin of the subject is indicative of the depth of penetration of the at least one micro-needle into the skin of the subject.

Preferably, the measuring electrodes are located on one of the urging means and the micro-needle element.

In one embodiment of the invention the measuring electrodes are located on the micro-needle element. Alternatively, the measuring electrodes are located on the urging means.

Preferably, each measuring electrode comprises a pointed element.

Advantageously, each measuring electrode comprises a micro-needle. Alternatively, each measuring electrode is of an electrically conductive material. Preferably, each measuring electrode is of length substantially similar to the length of the at least one micro-needle, and preferably, each measuring electrode is of length relative to the length of the at least one micro-needle so that the measuring electrodes penetrate the skin of the subject just prior to penetration of the skin of the subject by the at least one micro-needle, and advantageously, each measuring electrode is of length at least 10% longer than the at least one micro-needle, and preferably, in the range of 20% to 35% longer than the at least one micro-needle, and advantageously, in the range of 30% to 33% longer than the at least one micro-needle.

Preferably, the impedance detecting means is located on one of the urging means and the micro-needle element.

In one embodiment of the invention the impedance detecting means is located on the urging means. Alternatively, the impedance detecting means is located on the micro-needle element.

In one embodiment of the invention the determining means for determining when the at least one micro-needle penetrates the skin of the subject to the desired depth comprises a pressure sensing means for detecting the pressure under which the micro-needle element is urged into engagement with the skin of the subject. Preferably, the pressure sensing means comprises a piezoelectric crystal. Alternatively, the pressure sensing means comprises a strain gauge.

Preferably, the pressure sensing means is located on one of the urging means and the micro-needle element.

In one embodiment of the invention the pressure sensing means is located on the urging means. Alternatively, the pressure sensing means is located on the micro-needle element.

Ideally, the pressure sensing means is located between the urging means and the micro-needle element.

In one embodiment of the invention the indicating means is responsive to the pressure sensing means detecting that the pressure with which the micro-needle element is urged into engagement with the skin of the subject is sufficient for urging the at least one micro-needle of the micro-needle element to penetrate the skin of the subject to the desired depth for producing the first human sensory perceptible signal.

Preferably, the indicating means is responsive to the pressure sensing means detecting when the pressure with which the micro-needle element is urged into engagement with the skin of the subject is sufficient for maintaining the at least one micro-needle penetrating the skin of the subject to the desired depth for producing a second human sensory perceptible signal. Advantageously, the indicating means is responsive to the pressure sensing means detecting that the pressure with which the micro-needle element is being urged into engagement with the skin of the subject is approaching the pressure which should be sufficient to urge the at least one micro-needle to penetrate the skin to the desired depth for producing a third human sensory perceptible signal.

Preferably, the indicating means is responsive to the pressure sensing means detecting that the pressure with which the micro-needle element is being urged into engagement with the skin of the subject is insufficient for penetrating the skin of the subject by the at least one micro-needle for producing a fourth human sensory perceptible signal.

In one embodiment of the invention the indicating means comprises a visual display means.

In another embodiment of the invention each human sensory perceptible signal produced by the indicating means is one of a visually perceptible signal and an aurally perceptible signal. Preferably, each human sensory perceptible signal produced by the indicating means is a visually perceptible signal. Advantageously, the first human sensory perceptible signal is provided by activating a light of a first colour. Advantageously, the second human sensory perceptible signal is provided by activating a light of a second colour. Preferably, the third human sensory perceptible signal is provided by activating the light of one of the first and second colours to flash or by activating a light of a different colour to that of the first and second colours. Advantageously, the fourth human sensory perceptible signal is provided by activating a light of a different colour to that of the first and second colours.

In one embodiment of the invention each light is provided by a light emitting diode.

Preferably, each light is located on one of the urging means and the micro-needle element. Advantageously, each light is located on the urging means.

In another embodiment of the invention a monitoring means is provided for detecting disengagement of the micro-needle element from the skin of the subject.

Preferably, the indicating means is responsive to the monitoring means detecting disengagement of the micro-needle element from the skin of the subject for producing a fifth human sensory perceptible signal indicating disengagement of the micro-needle device. Advantageously, the fifth human sensory perceptible signal is one of a visually and an aurally perceptible signal.

In a further embodiment of the invention the monitoring means is responsive to disengagement of the micro-needle element from the skin of the subject for disabling a medicament delivery means of the micro-needle element.

In another embodiment of the invention the means for determining when the at least one micro-needle of the micro-needle element has penetrated the skin of the subject to the desired depth is adapted for detecting disengagement of the micro-needle element from the skin of the subject.

In a further embodiment of the invention the monitoring means is responsive to the determining means determining disengagement of the micro-needle element from the skin of the subject.

In one embodiment of the invention the monitoring means is responsive to the pressure sensing means detecting pressure indicative of disengagement of the micro-needle element from the skin of the subject. Alternatively, the monitoring means is responsive to the electrical impedance detecting means detecting electrical impedance between the measuring electrodes being indicative of disengagement of the micro-needle element from the skin of the subject.

Preferably, the urging means is adapted for retaining the micro-needle element on the skin of the subject with the at least one micro-needle penetrating through the skin of the subject. Advantageously, the urging means is adapted for carrying the micro-needle element between the urging means and the skin of the subject.

Preferably, the urging means comprises one of a strap and a patch. In one embodiment of the invention the one of the strap and the patch is adapted for carrying the micro-needle element between the one of the strap and the patch and the skin of the subject.

In another embodiment of the invention the urging means comprises a patch. Preferably, the patch comprises a means for securing the patch to the skin of the subject. Advantageously, the patch comprises a self-adhesive coating adapted for securing the patch to the skin of the subject.

In an alternative embodiment of the invention the urging means comprises a strap. Preferably, the strap is adapted for extending around one of a limb or a trunk of the subject.

Preferably, an adjusting means is provided for adjusting tension in the strap for varying the pressure with which the micro-needle element is urged into engagement with the skin of the subject. Advantageously, the adjusting means comprises an adjustable clasp for adjustably securing the strap to the subject.

In one embodiment of the invention an electrical power supply means is located on one of the micro-needle element and the strap, and an electrically conducting means extends between the micro-needle element and the strap for conducting electrical power from the power supply means from the one of the micro-needle element and the strap, to the other of the micro-needle element and the strap.

In another embodiment of the invention the determining means for determining when the at least one micro-needle of the micro-needle element penetrates the skin of the subject to the desired depth is located on the strap, and the determining means is powered by the electrical power supply means through the electrical conducting means.

In one embodiment of the invention the electrical power supply means is located on the micro-needle element and the determining means is powered by the electrical power supply means through the electrical conducting means. Alternatively, the electrical power supply means is located on the strap, and the micro-needle element is powered by the electrical power supply means through the electrical conducting means.

In a further embodiment of the invention the determining means comprises the pressure sensing means and the electrical impedance detecting means.

In another embodiment of the invention the apparatus comprises the micro-needle element.

Preferably, the micro-needle element comprises a plurality of micro-needles. Advantageously, the micro-needle element is programmable for sequentially dispensing doses of one or more medicaments therefrom.

In another embodiment of the invention a means for recording each dose of the one or more medicaments and the time of administering of the dose is provided for subsequent analysis. Preferably, the means for recording each dose and the time of administering the dose is provided on one of the urging means and the micro-needle element. Advantageously, the means for recording each dose and the time of administering the dose is provided on the micro-needle element.

In an alternative embodiment of the invention the apparatus comprises an applicator for applying the micro-needle element to the skin of the subject, the applicator comprising a housing, and the urging means is located in the housing.

Preferably, the determining means for determining when the at least one micro-needle of the micro-needle element penetrates the skin of the subject to the desired depth is located on the applicator. Advantageously, the urging means comprises a piston slideable within the applicator. Advantageously, the piston is powered by one of a pneumatic powering means, an electrical powering means and a spring urging means.

In another embodiment of the invention the indicating means is provided on the applicator.

The invention also provides a micro-needle device comprising a micro-needle element having at least one micro-needle, an urging means for urging the micro-needle element into engagement with the skin of a subject and for retaining the micro-needle element in engagement with the skin of the subject with the at least one micro-needle penetrating the skin of the subject, a means for determining when the at least one micro-needle has penetrated the skin of the subject to a desired depth, and an indicating means responsive to the determining means for indicating when the at least one micro-needle has penetrated the skin of the subject to the desired depth.

In one embodiment of the invention the strap is adapted to carry a carrier housing, and the micro-needle element is carried in the carrier housing. Preferably, the micro-needle element is releasably carried in the carrier housing. Advantageously, the determining means is mounted in the carrier housing.

Preferably, the pressure sensing means located between the carrier housing and the micro-needle element.

In another embodiment of the invention the measuring electrodes are located on the carrier housing. Advantageously, the measuring electrodes are located on the carrier housing with the micro-needle element located therebetween.

Additionally the invention provides an applicator for applying a micro-needle element comprising at least one micro-needle to the skin of a subject, the applicator comprising a housing defining an abutment surface for abutting the micro-needle element, an urging means located in the housing for engaging the micro-needle element, the urging means being adapted to impart a pressure to the micro-needle element sufficient for urging the micro-needle element into engagement with the skin of the subject with the at least one micro-needle penetrating the skin of the subject to a desired depth when the micro-needle element is abutting the abutment surface.

In one embodiment of the invention the urging means acts through the abutment surface. In another embodiment of the invention the urging means comprises a spring. Preferably, the urging means comprises a compression spring.

In another embodiment of the invention the urging means is adapted to act between the housing and the micro-needle element. Preferably, a bore extends into the housing from the abutment surface for accommodating the urging means therein.

Further the invention provides a method for applying a micro-needle element having at least one micro-needle to a site on the skin of a subject, the method comprising urging the micro-needle element into engagement with the skin of the subject with the at least one micro-needle thereof penetrating the skin, determining when the at least one micro-needle penetrates the skin to a desired depth, and producing a first human sensory perceptible signal when the at least one micro-needle has penetrated the skin of the subject to the desired depth.

In one embodiment of the invention determining when the at least one micro-needle penetrates the skin to a desired depth is carried out by determining the electrical impedance between a pair of measuring electrodes adapted to penetrate the skin of the subject with the at least one micro-needle.

In another embodiment of the invention determining when the at least one micro-needle penetrates the skin to the desired depth is carried out by sensing the pressure with which the micro-needle element is urged into engagement with the skin of the subject.

Preferably, the first human sensory perceptible signal is produced when the pressure with which the micro-needle element is being urged into engagement with the skin of the subject is sufficient to urge the at least one micro-needle of the micro-needle element to penetrate the skin of the subject to the desired depth.

Advantageously, a second human sensory perceptible signal is produced when the pressure with which the micro-needle element is being urged into engagement with the skin of the subject is sufficient for maintaining the at least one micro-needle penetrating the skin of the subject to the desired depth.

Preferably, a third human sensory perceptible signal is produced when the pressure with which the micro-needle element is being urged into engagement with the skin of the subject is approaching the pressure which should be sufficient to urge the at least one micro-needle to penetrate the skin to the desired depth.

Advantageously, a fourth human sensory perceptible signal is produced when the pressure with which the micro-needle element is being urged into engagement with the skin of the subject is insufficient to urge the at least one micro-needle to penetrate the skin of the subject.

In one embodiment of the invention each human sensory perceptible signal is displayed on a visual display means.

In a further embodiment of the invention each human sensory perceptible signal is produced as one of a visually perceptible signal and an aurally perceptible signal. Preferably, each human sensory perceptible signal is produced as a visually perceptible signal.

Preferably, disengagement of the micro-needle element from the skin of the subject is detected.

In one embodiment of the invention disengagement of the micro-needle element from the skin of the subject is detected by detecting the electrical impedance between the measuring electrodes.

In another embodiment of the invention disengagement of the micro-needle element from the skin of the subject is detected by detecting the pressure with which the micro-needle element is being urged into engagement with the skin of the subject.

Preferably, delivery of a medicament from the micro-needle element is disabled in response to detection of the micro-needle element disengaging the skin of the subject.

In one embodiment of the invention the micro-needle element is urged into engagement with the skin of the subject by a strap. Preferably, the strap is adapted to extend around a limb or trunk of the subject.

In another embodiment of the invention the micro-needle element is urged into engagement with the skin of the subject by an applicator.

In a further embodiment of the invention the micro-needle element is urged into engagement with the skin of the subject by a patch. Preferably, the patch is a self-adhesive patch.

The invention further provides a device for retaining a micro-needle element in engagement with the skin of a subject with micro-needles of the micro-needle element penetrating the skin of the subject, the device comprising a securing means for securing the micro-needle element to the skin of the subject, and a detecting means for detecting disengagement of the micro-needle element from the skin of the subject.

In one embodiment of the invention the detecting means comprises a pair of spaced apart measuring electrodes adapted to penetrate the skin of the subject, and a means for determining the impedance between the measuring electrodes, the measuring electrodes being adapted to remain in penetrating engagement with the skin of the subject while the micro-needles remain in penetrating engagement with the skin of the subject.

Preferably, a means is provided for comparing the impedance between the measuring electrodes with a reference impedance to detect disengagement of the micro-needle element from the skin of the subject.

Advantageously, the measuring electrodes are located on the securing means adjacent the micro-needle element.

Preferably, the detecting means is adapted to output a disabling signal for disabling discharge of a medicament from the micro-needle device in response to detection of disengagement of the micro-needle element from the subject.

In one embodiment of the invention the securing means comprises a self-adhesive patch.

In another embodiment of the invention the securing means comprises a strap.

In a further embodiment of the invention the device comprises a micro-needle element, and the micro-needle element is responsive to the disabling signal for disabling discharge of a medicament from the micro-needle element.

The invention also provides a method for retaining a micro-needle element in engagement with the skin of a subject with micro-needles of the micro-needle element penetrating the skin of the subject, the method comprising securing the micro-needle element to the skin of the subject with a securing means, and detecting disengagement of the micro-needle element from the skin of the subject by a detecting means.

The advantages of the invention are many. A particularly important advantage of the invention is that the apparatus according to the invention applies the micro-needle element to the skin of the subject with the micro-needles of the micro-needle element penetrating the skin of the subject to a desired depth. Furthermore, in cases where the urging means of the apparatus for urging the micro-needle element into engagement with the skin of the subject is provided by a strap which surrounds a limb or trunk of the subject; as well as applying the micro-needle element to the skin of the subject with the micro-needles penetrating the skin of the subject to a desired depth, the apparatus also maintains the micro-needle element engaging the skin of the subject with the micro-needles penetrating the skin to the desired depth. This advantage is also achieved when the micro-needle element is urged into engagement with the skin of the subject by an urging means provided by an adhesive patch when the patch is secured to the skin of the subject.

A further advantage of the invention is achieved by providing the apparatus in the form of an applicator. Since the applicator comprises a means for determining when the at least one or more micro-needles have penetrated the skin to a desired depth, by applying the micro-needle device to a site on the skin of a subject with the applicator, the micro-needle device can be precisely engaged with the skin of the subject with the micro-needle or micro-needles thereof engaging the skin of the subject to the correct desired depth, thereby eliminating trial and error required when a micro-needle device is being applied to a site on the skin of the subject manually.

A further advantage of the invention is achieved when the apparatus is provided with a monitoring means for monitoring for disengagement of the micro-needle element from the skin of the subject, and in particular, for detecting disengagement of the micro-needles of the micro-needle element from the skin of the subject. Such disengagement may result from loosening of the urging means where the urging means is provided by a strap or a patch or by physical removal of the micro-needle element together with the urging means, such as the strap or the patch. By providing the monitoring means for monitoring for disengagement of the micro-needle element from the skin of the subject, the monitoring means may be adapted for disabling a programmable electronic circuit of the micro-needle element, thereby disabling discharge of the medicament or medicaments from the micro-needle element. A further advantage of the invention is achieved by recording the doses of the medicament and the times at which the doses are administered to the subject, since periods during which the micro-needle element is disengaged from the skin of the subject can be determined, and adherence to a particular administrative regime of the medicaments can be likewise determined.

The invention will be more clearly understood from the following description of some preferred embodiments thereof, which are given by way of example only, with reference to the accompanying drawings, in which:

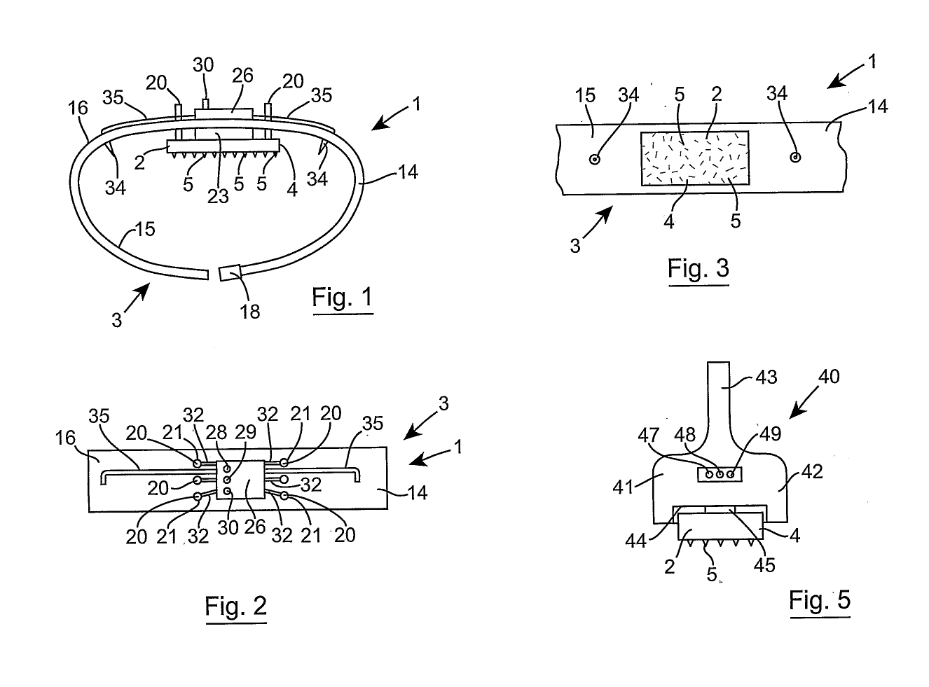

FIG. 1 is a side elevational view of apparatus according to the invention for applying a micro-needle element to a site on the skin of a subject,

FIG. 2 is a top plan view of the apparatus of FIG. 1,

FIG. 3 is an underneath plan view of a portion of the apparatus of FIG. 1,

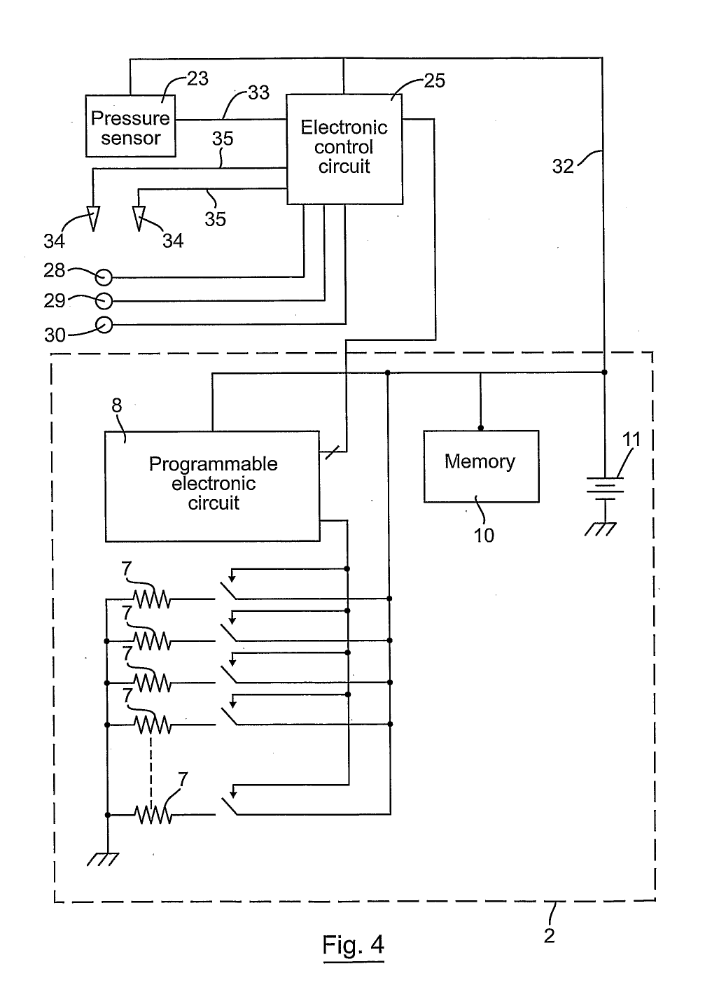

FIG. 4 is a circuit diagram of the apparatus of FIG. 1,

FIG. 5 is a front elevational view of apparatus according to another embodiment of the invention for applying a micro-needle element to a site on the skin of a subject,

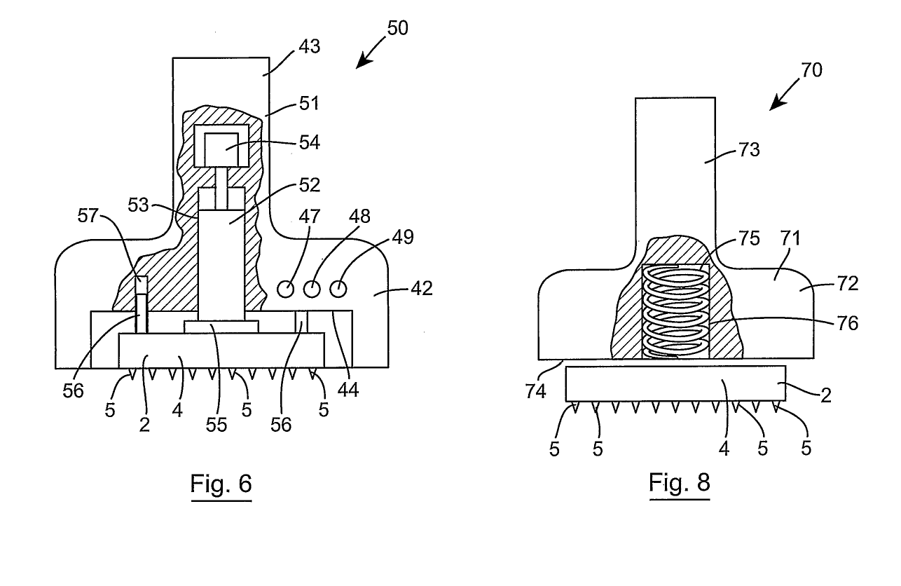

FIG. 6 is a front elevational view of apparatus according to another embodiment of the invention for applying a micro-needle element to a site on the skin of a subject,

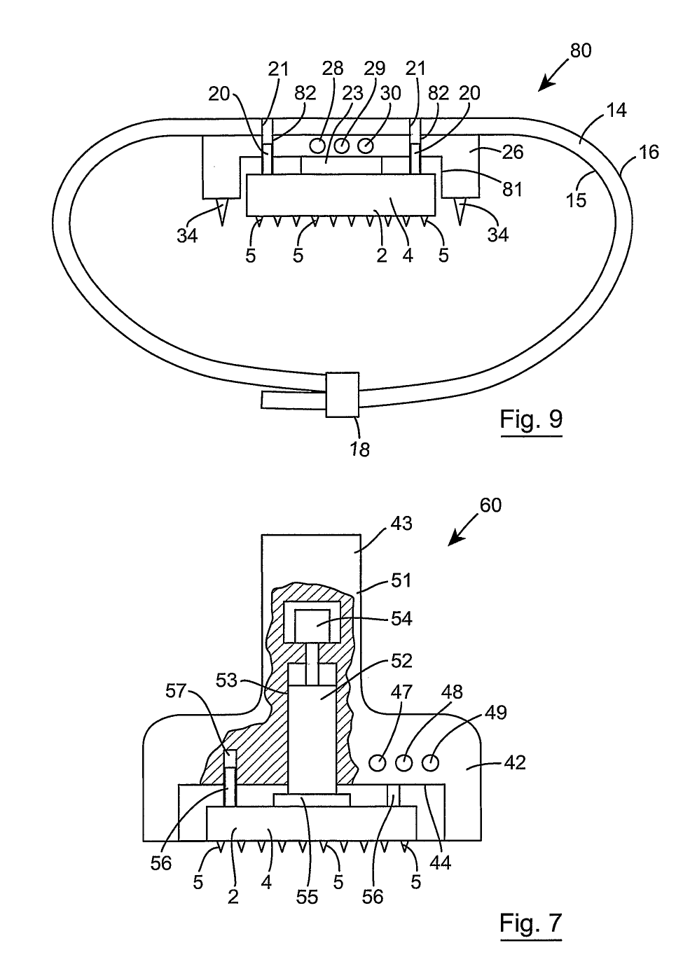

FIG. 7 is a front elevational view of apparatus according to a further embodiment of the invention for applying a micro-needle element to a site on the skin of a subject,

FIG. 8 is a front elevational view of apparatus according to a further embodiment of the invention for applying a micro-needle element to a site on the skin of a subject,

FIG. 9 is a side elevational view of apparatus according to a still further embodiment of the invention for applying a micro-needle element to a site on the skin of a subject,

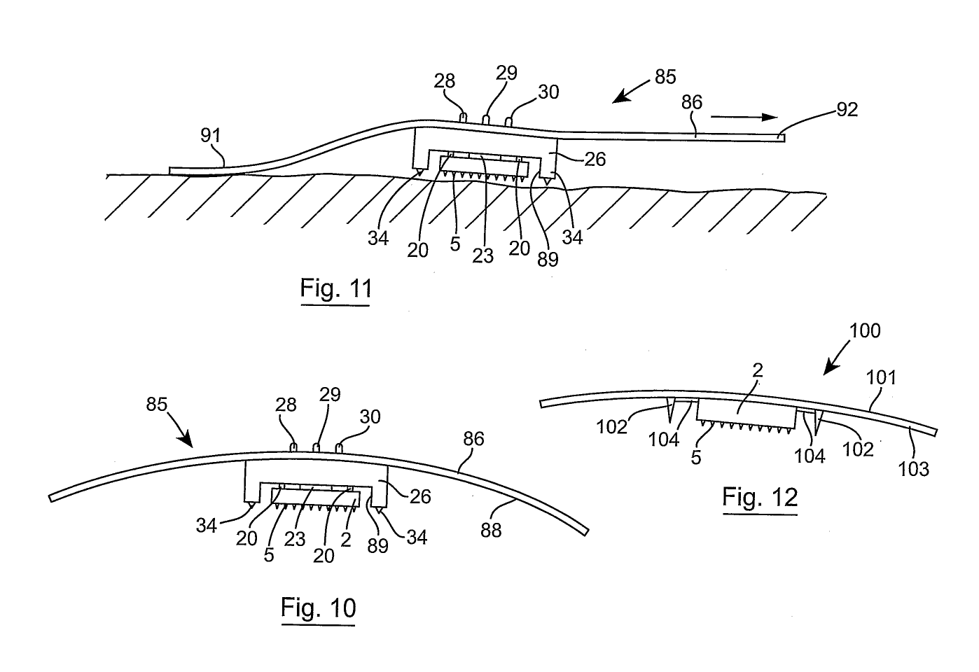

FIG. 10 is a side elevational view of apparatus according to a further embodiment of the invention for applying a micro-needle element to a site on the skin of a subject,

FIG. 11 is a side elevational view of the apparatus of FIG. 10, in use, and

FIG. 12 is a side elevational view of apparatus according to a further embodiment of the invention for retaining a micro-needle element on a site on the skin of a subject.

Referring to the drawings and initially to FIGS. 1 to 4 thereof, there is illustrated apparatus according to the invention, indicated generally by the reference numeral 1, for applying a micro-needle element 2 to a site on the skin of a subject. In this embodiment of the invention the apparatus 1 comprises a micro-needle device 3 also according to the invention. The micro-needle device 3 comprises the micro-needle element 2, which comprises a housing 4 typically of a polymer material having at least one micro-needle, and in this embodiment of the invention a plurality of micro-needles 5 extending therefrom of length of approximately 0.2 mm. The micro-needles 5 are arranged in a matrix which may comprise any number of micro-needles 5, and in this case the matrix is a six by six micro-needle matrix comprising a total of thirty-six micro-needles. Typically, the micro-needle device when viewed in plan is of area 10 mm by 10 mm, and a more typical preferred area is 20 mm by 20 mm.

A plurality of active substance reservoirs (not shown) for one or more medicaments are located in the housing 4, and typically one active substance reservoir is provided for each needle, with each active substance reservoir communicating with a corresponding one of the micro-needles 5 through which the medicament is discharged from the reservoir (not shown). The active substance reservoirs are arranged in a matrix similar to that of the micro-needles 5. A discharge means (also not shown) for individually discharging the medicaments from the respective reservoirs (not shown) through the corresponding ones of the micro-needles 5 is also provided in the housing 4. Any suitable discharge means may be provided, however, in this embodiment of the invention the discharge means comprises a plurality of drive substance reservoirs, one drive substance reservoir being provided for each active substance reservoir. A drive substance, which typically is an expandable substance which expands on being subjected to heat, is located in the drive substance reservoirs for driving the active substance from the active substance reservoirs through the micro-needles. A typical drive substance comprises a plurality of gas filled micro-spheres; such a substance is sold under the Trade Mark EXPANCEL. The drive substance reservoirs (not shown) are arranged in a matrix similar to that of the micro-needles and are aligned with the active substance reservoirs.

An activating means comprising a plurality of electrically powered heating elements 7 are provided in the housing 4, one heating element 7 being provided for each drive substance reservoir, for raising the temperature of the drive substance in the corresponding drive substance reservoir (not shown) for expanding the drive substance, for in turn discharging the medicament from the corresponding active substance reservoir through the corresponding micro-needle. The heating elements 7 are provided in a matrix similar to that of the micro-needles and are aligned with the drive substance reservoirs. Typical micro-needle elements are disclosed in PCT Published Application Specifications Nos. WO 2006/060106 and WO 2008/012788.

In this embodiment of the invention the micro-needle element 2 comprises a programmable electronic circuit 8 which is programmable for facilitating selective and sequential discharging of medicaments from the active substance reservoirs (not shown) of the housing 4 through the micro-needles 5. The programmable electronic circuit 8 of the micro-needle element 2 is programmed to discharge the one or more medicaments in the active substance reservoirs (not shown) at predefined times and at predefined intervals. Additionally, the programmable electronic circuit 8 of the micro-needle element 2 is programmed to record particulars of the medicaments and the times of discharge of the medicaments to the subject in a memory 10 for subsequent analysis. The micro-needle element 2 also comprises an in-built power supply 11 in the housing 4 for powering the heating elements 7 and for powering the programmable electronic circuit 8. In this embodiment of the invention the power supply 11 is implemented in the housing 4 in similar fashion as the power supply is implemented in the micro-needle element disclosed in PCT Published Application. Specification No. WO 2006/060106.

The micro-needle device 3 is particularly suitable for securing the micro-needle element 2 to a site on an arm of the subject, and comprises an urging means, namely, a strap 14 having an inner side 15 and an outer side 16 for urging the micro-needle element 2 into engagement with the skin on the arm of a subject with the micro-needles 5 penetrating the skin. The micro-needle element 2 is located on the inner side 15 of the strap 14, so that when the strap 14 is secured around the arm of a subject, the micro-needle element 2 is located between the strap 14 and the skin of the subject with the micro-needles 5 penetrating the skin. A securing means which also includes an adjusting means comprising an adjustable clasp 18 is provided on the strap 14 for securing the strap 14 around the arm of the subject, and for adjusting the tension in the strap 14 as the strap 14 is being tightened around the arm of the subject, and in turn the tension in the strap 14. By adjusting the tension in the strap 14, the pressure with which the micro-needle element 2 is urged into engagement with the skin of the subject is likewise adjusted.

Three electrically conductive guide pins 20 extend upwardly from the micro-needle element 2 on respective opposite ends thereof and slideably engage corresponding guide bores 21 in the strap 14 for facilitating movement of the micro-needle element 2 towards and away from the strap 14.

A determining means, namely, a first determining means for determining when the micro-needles 5 of the micro-needle element 2 penetrate the skin of the subject to a desired depth, in this embodiment of the invention, comprises a pressure sensing means, namely, a pressure sensor 23 located between the micro-needle element 2 and the strap 14. The pressure sensor 23 monitors the pressure with which the strap 14 urges the micro-needle element 2 into engagement with the skin of the subject. In this embodiment of the invention the pressure sensor 23 comprises a piezoelectric crystal, although any other suitable pressure sensing means may be provided, for example, a strain gauge. The pressure sensor 23 is secured to the strap 14 on the inner side 15 thereof and abuts the micro-needle element 2.

An electronic control circuit 25 in a carrier housing 26 located on the strap 14 on the outer side 16 thereof above the pressure sensor 23 monitors signals from the pressure sensor 23. The signals from the pressure sensor 23 are indicative of the pressure with which the micro-needle element 2 is being urged by the strap 14 into engagement with the skin of the subject, and in turn are indicative of the depth of penetration or otherwise of the skin of the subject by the micro-needles 5.

An indicating means comprising three light emitting diodes, namely, a first green light emitting diode 28, a second amber light emitting diode 29 and a third red light emitting diode 30 are located in the housing 26, and are operable under the control of the electronic control circuit 25 in response to the pressure read by the electronic control circuit 25 from the pressure sensor 23 to indicate by means of visually perceptible signals the pressure with which the micro-needle element 2 is being urged into engagement with the skin of the subject by the strap 14. The first green light emitting diode 28 is operated and powered continuously to produce a first signal in response to the pressure read from the pressure sensor 23 being indicative of the pressure applied to the micro-needle element 2 by the strap 14 being sufficient to urge the micro-needles 5 to penetrate the skin of the subject to the desired depth.

Once the micro-needles 5 have penetrated the skin of the subject to the desired depth, the pressure required to maintain the micro-needle element 2 in engagement with the skin of the subject with the micro-needles 5 penetrating the skin to the desired depth is less than that required to initially urge the micro-needles 5 to penetrate the skin to the desired depth. Thus, once the micro-needle element 2 has been urged into engagement with the skin of the subject so that the micro-needles 5 penetrate the skin of the subject to the desired depth, the pressure with which the micro-needle element 2 is urged into engagement with the skin of the subject may be relaxed. Thus, with the first light emitting diode 28 continuously powered, indicating that the micro-needles 5 have been urged to penetrate the skin to the desired depth, the clasp 18 of the strap 14 is adjusted to release the pressure with which the micro-needle element 2 is being urged by the strap 14 into engagement with the skin until the first light emitting diode 28 is powered down and the pressure read by the electronic control circuit 25 from the pressure sensor 23 is indicative of the pressure which is sufficient for maintaining the micro-needles 5 penetrating the skin of the subject to the desired depth. At which stage the electronic control circuit 25 operates the second light emitting diode 29 continuously to produce a second signal, thereby indicating that the pressure with which the micro-needle element 2 is being urged into engagement with the skin of the subject by the strap 14 is sufficient for maintaining the micro-needles 5 penetrating the skin of the subject to the desired depth. For so long as the pressure being exerted by the strap 14 on the micro-needle element 2 is sufficient to maintain the micro-needles 5 penetrating the skin to the desired depth, the second light emitting diode 29 remains continuously powered up by the electronic control circuit 25.

The electronic control circuit 25 also operates the second light emitting diode 29 to flash to produce a third signal, which indicates that the pressure with which the strap 14 is being initially tightened is approaching the pressure required to urge the micro-needle element 2 into engagement with the skin of the subject.

When the strap 14 is initially being tightened, the electronic control circuit 25 powers up the third light emitting diode 30 to produce a fourth signal until the pressure at which the micro-needle element 2 is being urged into engagement with the skin of the subject is approaching the pressure which is required in order that the micro-needles 5 penetrate the skin of the subject to the desired depth. Once the pressure being exerted on the micro-needle element 2 by the strap 14 is approaching that which is sufficient to urge the micro-needles 5 to penetrate the skin to the desired depth, the electronic control circuit 25 powers down the third light emitting diode 30 and operates the second light emitting diode 29 to flash amber to produce the third signal.

Thereafter the second light emitting diode 29 is operated by the electronic control circuit 25 to flash amber until the pressure with which the micro-needle element 2 is being urged into engagement with the skin of the subject is sufficient to cause the micro-needles 5 to penetrate the skin of the subject to the desired depth. At which stage the electronic control circuit 25 powers up the first light emitting diode 28 continuously until the pressure with which the micro-needle element 2 is being urged by the strap 14 into engagement with the skin of the subject is relaxed. When the pressure with which the micro-needle element 2 is being urged by the strap 14 into engagement with the skin is relaxed to the state which is sufficient for maintaining the micro-needles 5 penetrating the skin of the subject to the desired depth, the electronic control circuit 25 powers down the first light emitting diode 28 and powers up the second light emitting diode 29 to continuously glow amber, as described above.

During normal use of the micro-needle device 3, should the pressure exerted by the strap 14 on the micro-needle element 2 fall below the pressure required to maintain the micro-needles 5 penetrating the skin of the subject to the desired depth, the second light emitting diode 29 is operated by the electronic control circuit 25 to flash, thus indicating that tightening of the strap 14 is required. On the strap 14 being tightened to apply the pressure to the micro-needle element 2 which is sufficient for maintaining the micro-needles 5 penetrating the skin of the subject to the desired depth, the electronic control circuit 25 operates the second light emitting diode 29 in the continuously powered up mode.

Should the pressure exerted by the strap 14 on the micro-needle element 2 fall below a pressure which is approaching the pressure which is required in order to urge the micro-needles 5 to penetrate the skin of the subject, the second light emitting diode 29 is powered down and the third light emitting diode 30 is powered up continuously by the electronic control circuit 25, thus indicating tightening of the strap 14 is required. On tightening of the strap 14, once the pressure exerted by the strap 14 on the micro-needle element 2 begins to approach that which is necessary to penetrate the micro-needles 5 through the skin of the subject to the desired depth, the third light emitting diode 30 is powered down and the second light emitting diode 29 is operated by the electronic control circuit 25 to flash amber. This continues until the strap 14 has been tightened to apply the pressure to the micro-needle element 2 which is sufficient for maintaining the micro-needles 5 penetrating the skin of the subject to the desired depth. At which stage the electronic control circuit 25 powers up the second light emitting diode 29 continuously.

The electronic control circuit 25 is programmed to compare the values of the signals read from the pressure sensor 23 with appropriate reference values which correspond to ideal pressures with which the micro-needle element 2 should be urged by the strap 14 into engagement with the skin of the subject during securing of the micro-needle device 3 to the subject, and then during use of the micro-needle device 3. The electronic control circuit is programmable to facilitate entering of the reference values which correspond to the ideal pressure values. The ideal pressure values and in turn the reference values will vary depending on the site of the subject to which the micro-needle element is to be attached.

Electrical power is supplied to the electronic control circuit 25 in the housing 26, to the pressure sensor 23 and to the light emitting diodes 28, 29 and 30 from the in-built power supply 11 of the micro-needle element 2 through the electrically conductive guide pins 20. The guide bores 21 extending through the strap 14 are internally coated with an electrically conductive coating, through which electrical power is conducted from the guide pins 20. Electrically conductive tracks 32 from the electrically conductive coating on the bores 21 provide electrical continuity from the guide pins 20 to the electronic control circuit 25 in the housing 26. The pressure sensor 23 is powered by electrically conductive tracks 33 extending through the strap 14 from the housing 26 to the pressure sensor 23.

The means for determining when the micro-needles 5 penetrate the skin to the desired depth also comprises a second determining means, which in this case is adapted for determining disengagement of the micro-needle element 2 from the subject, and comprises an impedance detecting means provided by a pair of spaced apart pointed electrically conductive measuring electrodes 34 for penetrating the skin of the subject to detect the electrical impedance beneath the skin of the subject. The measuring electrodes 34 are located on the inner side 15 of the strap 14 on respective opposite sides of the micro-needle element 2 and are adapted to penetrate the skin of the subject while the pressure applied by the strap 14 on the micro-needle element 2 is sufficient for maintaining the micro-needles 5 penetrating the skin of the subject to the desired depth. The measuring electrodes 34 in this embodiment of the invention are of dimensions substantially similar to the dimensions of the micro-needles 5 and are each approximately 0.2 mm in length. Electrically conductive tracks 35 on the strap 14 electrically connect the measuring electrodes 35 to the electronic control circuit 25 located in the housing 26. The electronic control circuit 25 is programmed to act as a monitoring means for monitoring the electrical impedance between the measuring electrodes 34.

The electronic control circuit 25 located in the housing 26 is programmed to compare the monitored impedance between the electrodes 34 with a predefined reference impedance which corresponds with the value of the impedance which would be detected between the electrodes 34, if the electrodes 34 were penetrating the skin of the subject with the micro-needles 5 penetrating the skin of the subject to the desired depth. When the impedance between the measuring electrodes monitored by the electronic control circuit 25 exceeds the predefined reference impedance indicating that the micro-needle element 2 and the micro-needles 5 have become disengaged from the skin of the subject, the electronic control circuit 25 in the housing 26 outputs a disable signal to the programmable electronic circuit 8 of the micro-needle element 2. The programmable electronic circuit 8 is responsive to the signal from the electronic control circuit 25 to disable discharge of the medicaments from the micro-needle element 2 during periods while the micro-needle element 2 is disengaged from the skin of the subject. The electronic control circuit 25 on detecting the impedance between the measuring electrodes 34 being indicative of disengagement of the micro-needle element 2 from the skin of the subject also powers up the third light emitting diode 30 to light continuously to produce a fifth human sensory perceptible signal, thereby indicating disengagement of the micro-needle element 2 from the skin of the subject.

In use, with the micro-needle element 2 attached to the strap 14 by the guide pins 20 of the micro-needle element 2 slideably engaged with the guide bores 21 of the strap 14, the micro-needle element 2 is placed on the skin of the arm of the subject at the desired site. The strap 14 is then extended around the arm and engaged with the clasp 8. The clasp 8 is adjusted for tightening the strap 7 around the arm of the subject. Initially the third light emitting diode 30 is powered up by the electronic control circuit 25, and the first and second light emitting diodes 28 and 29 are powered down. The third light emitting diode 30 continues to be powered up until the pressure sensed by the pressure sensor 23 with which the micro-needle element 2 is being urged by the strap 14 into engagement with the skin is approaching the pressure which is sufficient to cause the micro-needles 5 to penetrate the skin of the subject to the desired depth. At that stage the third light emitting diode 30 is powered down and the second light emitting diode 29 is operated to flash while the strap 14 is being further tightened. On the pressure sensor 23 detecting that the pressure with which the micro-needle element 2 is being urged by the strap 14 into engagement with the skin is sufficient to penetrate the micro-needles 5 through the skin of the subject to the desired depth, the second light emitting diode 29 is powered down, and the first light emitting diode 28 is powered up continuously, thus indicating that the strap 14 has been tightened to a sufficient degree, so that the micro-needles 5 penetrate the skin of the subject to the desired depth.

At this stage, once the micro-needles 5 have penetrated the skin of the subject to the desired depth, the strap 14 is slackened slightly until the pressure detected by the pressure sensor 23 is just sufficient to maintain the micro-needles 5 penetrating the skin of the subject to the desired depth. On the pressure detected by the pressure sensor 23 at which the micro-needle element 2 is being urged by the strap 14 into engagement with the skin of the subject being at the pressure which is sufficient for maintaining the micro-needles 5 penetrating the skin of the subject to the desired depth, the first light emitting diode 28 is powered down, and the second light emitting diode 29 is powered up to continuously operate. This thus indicates that the pressure with which the micro-needle element 2 is being urged by the strap 14 into engagement with the skin is sufficient for maintaining the micro-needles 5 penetrating the skin to the desired depth. Thereafter, for so long as the strap 14 remains tightened to the appropriate tightness for maintaining the micro-needles 5 penetrating the skin to the desired depth, the second light emitting diode 29 remains powered up.

Thereafter the micro-needle device 2 operates in conventional fashion with the medicaments being dispensed at appropriate times at appropriate time intervals under the control of the programmable electronic circuit 8. The doses of the medicaments and their times of dispensing are recorded and stored in the memory 10 under the control of the programmable electronic circuit 8 for subsequent analysis.

At any stage during normal use of the micro-needle device 3, should the strap 14 become loose, and the pressure applied by the strap 14 to the micro-needle element 2 fall below the pressure which is sufficient for maintaining the micro-needles 5 penetrating the skin of the subject to the desired depth, the second light emitting diode 29 is operated by the electronic control circuit 25 to flash. Should the pressure applied by the strap 14 to the micro-needle element 2 fall further and below a pressure which is approaching that which is sufficient for penetrating the micro-needles 5 through the skin of the subject to the desired depth, the second light emitting diode 29 is powered down, and the third light emitting diode 30 is powered up continuously. Retightening of the strap 7 operates the second and third light emitting diodes 29 and 30 as already described until the pressure applied by the strap 14 to the micro-needle element 2 is sufficient for maintaining the micro-needles 5 penetrating the skin of the subject to the desired depth. At which stage, the second light emitting diode 29 is continuously operated by the electronic control circuit 25.

For so long as the impedance between the measuring electrodes 34 remains at or below the predefined reference impedance, the micro-needle device 3 continues to operate normally. In the event that the electronic control circuit 25 in the housing 26 detects the impedance between the measuring electrodes 34 rising above the predefined reference impedance, thus indicating disengagement of the micro-needle element 2 from the skin of the subject, the electronic control circuit 25 in the housing 26 outputs the disable signal to operate the programmable electronic circuit 8 in the micro-needle element 2 to disable discharge of the medicaments from the micro-needle element 2, until the micro-needle device 3 is again returned and secured to the arm of the subject, and the impedance between the penetrating electrodes 34 is below the predefined reference impedance. At which stage the electronic control circuit 25 in the housing 26 outputs an enable signal to the programmable electronic circuit 8 in the housing 4 of the micro-needle element 2 to recommence normal operation of discharging the medicaments from the active substance reservoirs (not shown) at the predefined times at the predefined intervals, and recording and storing the doses of the medicaments and the times of dispensing of the respective doses of the medicaments in the memory 10. Additionally, on the electronic control circuit 25 detecting the impedance between the measuring electrodes 34 being indicative of disengagement of the micro-needle element 2 from the skin of the subject, the electronic control circuit 25 powers up the third light emitting diode 30 to light continuously to produce the fifth human sensory perceptible signal, thereby indicating disengagement of the micro-needle element 2 from the skin of the subject.

While the monitoring means for monitoring disengagement or removal of the micro-needle element 2 from the skin of the subject has been described as comprising the impedance detecting means which comprises the measuring electrodes 34, it is envisaged that the monitoring means may be provided by configuring the electronic control circuit 25 to compare the pressure read from the pressure sensor 23 with a predefined reference pressure which would be indicative of disengagement of the micro-needle element 2 from the skin of the subject. On the pressure read from the pressure sensor 23 falling below that predefined reference pressure, the electronic control circuit 25 would determine that the micro-needle element 2 had been disengaged or removed from the subject, and would output the disable signal to the programmable electronic circuit 8 in the micro-needle element 2 to disable discharge of the medicaments from the micro-needle element 2 while the micro-needle element 2 is disengaged or removed from the skin of the subject as already described with reference to the monitoring of the measuring electrodes 34.

It is also envisaged that the means for determining when the micro-needles 5 of the micro-needle element 2 have penetrated the skin to the desired depth could be provided by the impedance detecting means which comprises the measuring electrodes 34. In which case, the electronic control circuit 25 would be programmed to compare the impedance between the measuring electrodes 34 with appropriate predefined reference impedance values which would correspond to ideal impedance values which should be detected when the micro-needle element 2 is being urged by the strap 14 into engagement with the skin of the subject during securing of the micro-needle device 3 to the subject, and then during use of the micro-needle device 3 as already described. The impedance between the measuring electrodes 34 detected by the electronic control circuit would then be compared with the predefined reference impedance values, and the three light emitting diodes 28, 29 and 30 would be operated appropriately as already described during securing of the micro-needle device 3 to the subject and then during use of the micro-needle device 3.

It is also envisaged that the micro-needle device 3 may be provided with only one of the first and second determining means, either the pressure sensor or the measuring electrodes.

Additionally, while the micro-needle device 3 has been described as comprising the electrical power supply located in the micro-needle element 2 for powering the electronic control circuit 25 and the pressure sensor 23, as well as the measuring electrodes 34, it is envisaged that in certain cases the power supply may be provided either mounted on the strap 14 or located in the housing 26, and in which case, it is envisaged that the power supply would be provided by a battery. When the power supply is provided on the strap 14 or in the housing 26, power would be supplied to the micro-needle element 2 through the electrically conductive guide pins 20. Indeed, it is envisaged that it may be preferable to provide the power supply as a battery located either on the strap 14 or in the housing 26.

Referring now to FIG. 5, there is illustrated apparatus according to another embodiment of the invention, indicated generally by the reference numeral 40 for applying a micro-needle element 2 similar to the micro-needle element 2 of the micro-needle device 3 of FIGS. 1 to 4 to the skin of a subject with the micro-needles 5 penetrating the skin of the subject to a desired depth. In this embodiment of the invention the apparatus 40 comprises an applicator 41 which comprises a housing 42 having a handle 43 extending from the housing 42. The housing 42 defines an abutment surface 44 on which is mounted a means for determining when the micro-needles 5 of the micro-element 2 are penetrating the skin of the subject to the desired depth, which in this embodiment of the invention is a pressure sensor 45. The pressure sensor 45 is similar to the pressure sensor 23 of the micro-needle device 3. The pressure sensor 45 is adapted for engaging the micro-needle element 2 so that the micro-needle element 2 may be urged into engagement with the skin of the subject by the applicator 41. An electronic control circuit (not shown) but substantially similar to the electronic control circuit 25 in the housing 42 monitors the pressure read by the pressure sensor 45 and operates first, second and third light emitting diodes 47, 48 and 49 which are similar to the first, second and third light emitting diodes 28, 29 and 30, respectively, to indicate the pressure with which the micro-needle element 2 is being urged into engagement with the skin of the subject, in similar fashion as already described with reference to the micro-needle device 3 of FIGS. 1 to 4. In this embodiment of the invention a power supply provided by a battery (not shown) is located in the housing 42 of the applicator 41 for powering the electronic control circuit (not shown), the first, second and third light emitting diodes 47 to 49 and the pressure sensor 45.

In use, the micro-needle element 2 is placed on the skin of a subject at the desired site, and with the applicator 41 gripped by the handle 43, the applicator 41 is used to urge the micro-needle element 2 into engagement with the skin of the subject. The pressure sensor 45 monitors the pressure with which the micro-needle element 2 is being urged by the applicator 41 into engagement with the skin of the subject. Initially the third light emitting diode 49 is powered up. When the pressure detected by the pressure sensor 45 is approaching the pressure which is sufficient to cause the micro-needles 5 to penetrate the skin of the subject to the desired depth, the third light emitting diode 49 is powered down and the second light emitting diode 48 is operated to flash until the pressure detected by the pressure sensor 45 is sufficient for penetrating the micro-needles 5 through the skin of the subject to the desired depth. At which stage the second light emitting diode 48 is powered down and the first light emitting diode 47 is powered up. At that stage the applicator 41 is removed and the micro-needle element 3 is then held in contact with the skin of the subject by a suitable self-adhesive patch (not shown).

The apparatus 40 is particularly suitable for applying the micro-needle element 2 to the skin of the subject adjacent the abdomen of the subject, where, in general, it would not be feasible to secure the micro-needle element 2 by a strap similar to the strap 14.

Referring now to FIG. 6, there is illustrated apparatus according to another embodiment of the invention, indicated generally by the reference numeral 50, also for applying a micro-needle element 2 to the skin of a subject with the micro-needles 5 of the micro-needle element 2 penetrating the skin of the subject to a desired depth. The apparatus 50 is substantially similar to the apparatus 40 and similar components are identified by the same reference numerals. The apparatus 50 comprises an applicator 51, and the main difference between the apparatus 50 and the apparatus 40 is that an urging means, namely, a piston 52 is slideably mounted in a bore 53 in the housing 42 of the applicator 51 for urging the micro-needle element 2 into engagement with the skin of the subject. The piston 52 is powered by an electrically powered linear motor 54 mounted in the housing 42. The piston 52 terminates in a pressure sensor 55 similar to the pressure sensors 45 and 23 for detecting the pressure with which the micro-needle element 2 is being urged by the piston 52 into engagement with the skin of the subject.

In this embodiment of the invention an electronic control circuit (not shown) which is substantially similar to the electronic control circuit 25 of the micro-needle device 3 is located within the housing 42 of the applicator 51 and reads signals from the pressure sensor 55 for determining the pressure at which the micro-needle element 2 is being urged by the piston 52 into engagement with the skin of the subject. The electronic control circuit (not shown) operates the first, second and third light emitting diodes 47, 48 and 49 in a similar manner as described with reference to the apparatus 40 of FIG. 5. Guide pins 56, which are similar to the guide pins 20, slideably engage bores 57 in the housing 42 for guiding the micro-needle element 2 relative to the housing 42.

In this embodiment of the invention the electronic control circuit (not shown), the electrically powered linear motor 54 and the first, second and third light emitting diodes 47, 48 and 49 as well as the pressure sensor 55 are powered by a battery (not shown) located in the housing 42.

In use, the micro-needle element 2 is placed on the skin of the subject at the desired site and the applicator 51 of the apparatus 50 is placed over the micro-needle element 2 with the pressure sensor 55 engaging the micro-needle element 2. The linear motor 54 is then operated for urging the piston 52 downwardly in the bore 53, for in turn urging the micro-needle element 2 into engagement with the skin. The electronic control circuit (not shown) reads signals from the pressure sensor 55 for determining the pressure with which the micro-needle element 2 is being urged into engagement with the skin by the piston 52. The electronic control circuit (not shown) operates the light emitting diodes 47 to 49 as already described, and deactivates the motor 54 when the pressure read from the pressure sensor 55 has reached a pressure sufficient for urging the micro-needle element 2 into engagement with the skin of the subject with the micro-needles 5 penetrating the skin to the desired depth.

The guide pins 56 are then removed from the micro-needle element 2, and the micro-needle element 2 is secured to the skin of a subject by a suitable self-adhesive patch (not shown).

Referring now to FIG. 7, there is illustrated apparatus according to another embodiment of the invention, indicated generally by the reference numeral 60, also for applying a micro-needle element 2 to the skin of a subject with the micro-needles 5 of the micro-needle element 2 penetrating the skin of the subject to a desired depth. The apparatus 60 is substantially similar to the apparatus 50 described with reference to FIG. 6, and similar components are identified by the same reference numerals. The only difference between the apparatus 60 and the apparatus 50 is that the electronic control circuit (not shown), the linear motor 54, the pressure sensor 55 and the first, second and third light emitting diodes 47, 48 and 49 are powered by an electrical power supply (not shown) in the micro-needle element 2. Electrical power from the battery (not shown) in the micro-needle element 2 is conducted to the electronic control circuit (not shown) in the housing 42 through the guide pins 56 and the bores 57 in the housing 42.

Otherwise the apparatus 60 is similar to the apparatus 50 and its use is likewise similar.

Referring now to FIG. 8, there is illustrated apparatus 70 also according to another embodiment of the invention for applying a micro-needle element 2 to the skin of a subject. In this embodiment of the invention the apparatus 70 comprises an applicator 71 which comprises a housing 72 and a handle 73 extending from the housing 72. The housing 72 terminates in an abutment surface 74 for abutting the micro-needle element 2. A compression spring 75 is located in a bore 76 in the housing 72 and extends from the bore 76 through the abutment surface 74. The compression spring 75 acts between the housing 72 and the micro-needle element 2, and is of spring strength such that when the compression spring 75 has been compressed to the extent that the micro-needle element 2 abuts the abutment surface 74, the pressure which is applied to the micro-needle element 2 by the compression spring 75 is the appropriate pressure required to urge the micro-needle element 2 into engagement with the skin of the subject with the micro-needles 5 penetrating the skin of the subject to the desired depth. Accordingly, in this embodiment of the invention the abutment surface 74 acts as both the determining means and the indicating means to both determine and indicate when the micro-needle element 2 has been urged into engagement with the skin of the subject with the micro-needles 5 penetrating the skin to the desired depth.

It is envisaged that in certain cases, in this embodiment of the invention a sensing means may be provided in the housing 72 adjacent the abutment surface 74 for detecting precisely when the micro-needle element 2 is in abutment engagement with the abutment surface 74. Such a sensing means could be provided by a proximity sensor, an optical sensor, or any other suitable sensor.

In use, the micro-needle element 2 is placed on the skin of the subject at the desired site. The applicator 71 is then placed over the micro-needle element 2 with the compression spring 75 engaged with the micro-needle element 2. The applicator 71 is then urged downwardly for urging the micro-needle element 2 into engagement with the skin of the subject until the micro-needle element 2 just abuts the abutment surface 74, at which stage the micro-needle element 2 is being urged into engagement with the skin at the appropriate pressure so that the micro-needles 5 penetrate the skin of the subject to the desired depth. The applicator 71 is then removed and the micro-needle element 2 is retained in contact with the skin of the subject by a suitable self-adhesive patch.

Referring now to FIG. 9, there is illustrated a micro-needle device according to another embodiment of the invention, indicated generally by the reference numeral 80. The micro-needle device 80 is substantially similar to the micro-needle device 3 and similar components are identified by the same reference numerals. The main difference between the micro-needle device 80 and the micro-needle device 3 is that the carrier housing 26 is located on the inner side 15 of the strap 14 as opposed to on the outer side 16 thereof in the case of the micro-needle device 3, and the micro-needle element 2 is located in a recess 81 formed in the housing 26. A battery (not shown) in the housing 26 powers both the electronic control circuit (not shown) in the housing 26 and the micro-needle element 2. The guide pins 20 are slideable in bores 82 which extend through the carrier housing 26 and through the strap 14. In this embodiment of the invention electrical power is transferred from the electronic control circuit 25 to the micro-needle element 2 through the guide pins 20 and suitable brushes located in the carrier housing 26 bearing on the guide pins 20. Additionally, in this embodiment of the invention the measuring electrodes 34 are mounted on the carrier housing 26 on opposite sides of the recess 81.

Otherwise, the micro-needle device 80 and its use is similar to that of the micro-needle device 3.

Referring now to FIGS. 10 and 11, there is illustrated apparatus according to another embodiment of the invention, indicated generally by the reference numeral 85, for applying a micro-needle element 2 similar to the micro-needle element 2 of the apparatus 1 of FIGS. 1 to 4 to a site on the skin of a subject. The apparatus 85 is substantially similar to the apparatus 1 and similar components are identified by the same reference numerals. In this embodiment of the invention the apparatus 85 comprises a self-adhesive patch 86 which secures the micro-needle element 2 to the skin of the subject with the micro-needles 5 penetrating the skin of the subject to the desired depth. In this embodiment of the invention the strap 14 of the micro-needle device 3 of the apparatus 1 is replaced by the patch 86.

A carrier housing 26 similar to the housing 26 of the micro-needle device 3 is secured to a self-adhesive side 88 of the patch 86. The housing 26 defines a recess 89 within which the micro-needle element 2 is located. Guide pins 20 similar to the guide pins 20 of the micro-needle device 3 extend from the micro-needle element 2 and slideably engage corresponding guide bores (not shown) in the housing 26 for guiding the micro-needle element 2 relative to the housing 26. The guide bores (not shown) in the housing 26 are similar to those in the housing 26 of the micro-needle device 3′. A pressure sensor 23 similar to the pressure sensor 23 of the micro-needle device 3 is secured to the housing 26 in the recess 89 and abuts the micro-needle element 2 for detecting the pressure with which the micro-needle element 2 is being urged into engagement with the skin by the patch 86. The guide pins 20 engage electrical bushes in the guide bores (not shown) to provide electrical continuity between an electronic control circuit (not shown) in the housing 26 and the programmable electronic circuit (also not shown) in the micro-needle element 2, which is similar to that already described with reference to the micro-needle device 3. The electronic control circuit (not shown) in the housing 26 is similar to the electronic control circuit 25 of the micro-needle device 3.

Three light emitting diodes, namely, a first green light emitting diode 28, a second amber light emitting diode 29 and a third red light emitting diode 30 extend from the housing 26 through the patch 86 in order to indicate the pressure with which the micro-needle element 2 is being urged into engagement with the skin of the subject by the patch 86 in similar manner as described with reference to the light emitting diodes 28 to 30 of the micro-needle device 3.

A pair of measuring electrodes 34 similar to the measuring electrodes 34 of the micro-needle device 3 extend from the housing 26 on respective opposite sides of the recess 89, and are coupled to the electronic control circuit (not shown) in the housing 26 which measures the impedance between the measuring electrodes 34. In this embodiment of the invention the measuring electrodes 34 are slightly longer than the micro-needles 5 so that the measuring electrodes 34 engage the skin of the subject just before the micro-needles 5 are about to engage the skin of the subject. This gives an initial indication that the micro-needles 5 are about to engage the skin of the subject. The electronic control circuit (not shown) is programmed to compare the impedance between the measuring electrodes 34 with a reference impedance, the value of which is similar to that which would be detected between the measuring electrodes 34 when the measuring electrodes 34 have just commenced to penetrate the skin of the subject. When the electronic control circuit (not shown) determines that the impedance between the measuring electrodes 34 is such that the measuring electrodes 34 have just penetrated the skin of the subject, the electronic control circuit (not shown) powers down the third red light emitting diode 30 and powers up the second amber light emitting diode 29 to commence flashing to produce the third signal to indicate that the pressure with which the micro-needle element 2 is being urged into engagement with the skin of the subject by the patch 86 is approaching the pressure which is sufficient to urge the micro-needles 5 to penetrate the skin of the subject. Thereafter operation of the light emitting diodes 28, 29 and 30 by the electronic control circuit (not shown) in the housing 26 is similar to that described with reference to the micro-needle device 3 of the apparatus 1, and the light emitting diodes 28, 29 and 30 are operated by the electronic control circuit (not shown) in the housing 26 in response to signals from the pressure sensor 23. The electronic control circuit (not shown) is responsive to the impedance between the measuring electrodes 34 being indicative of disengagement of the micro-needle device 2 from the skin of the subject as already described with reference to the micro-needle device 3 of the apparatus 1.

In this embodiment of the invention the micro-needles are of length approximately 0.75 mm, and the length of the two measuring electrodes 34 are each approximately 1 mm. Thus, the measuring electrodes 34 will have penetrated the skin of the subject to a depth of approximately 0.25 mm prior to penetration of the skin of the subject by the micro-needles 5.

The patch 86 may be an elongated patch which would extend across the housing 26 from one side to the other and extend outwardly on respective opposite sides of the housing. Alternatively, the patch may be a circular or square patch which would be of an area considerably greater than the area of the housing 26, and would extend completely around and beyond the housing 26.

Otherwise, the apparatus 85 is similar to the apparatus 1.

In use, the micro-needle device 3 of the apparatus 85 is applied to the skin 90 of the subject as will now be described with reference to FIG. 11. Initially, a portion 91 of the patch 86 to one side of the housing 26 is secured to the skin of the subject. The other side 92 of the patch 86 is gripped and pulled in the direction of the arrow A to tension the patch 86. With the patch 86 tensioned, the side 92 of the patch 86 is then urged downwardly to the skin. The amount of tensioning of the patch 86 determines the pressure with which the patch 86 urges the micro-needle element 2 into engagement with the skin. When the patch 86 urges the micro-needle element 2 into engagement with the skin with the pressure which is sufficient that the micro-needles 5 penetrate the skin of the subject to the desired depth, the second light emitting diode 29 is powered down, and the first light emitting diode 28 is powered up. At that stage the tensioning of the patch 86 is slightly relaxed until the first light emitting diode 28 is powered down and the second light emitting diode is powered up. At that stage the side 92 of the patch 86 which is still gripped is then urged into engagement with the skin of the subject in order to retain the micro-needle element 2 in engagement with the skin of the subject at the desired pressure for maintaining the micro-needles 5 penetrating the skin of the subject.

Otherwise, the use of the apparatus 85 is similar to that of the apparatus 1.

Referring now to FIG. 12, there is illustrated a device also according to the invention, indicated generally by the reference numeral 100, for retaining a micro-needle element similar to the micro-needle element 2 in engagement with the skin of a subject after the micro-needle element has been applied to the skin of the subject with the micro-needles 5 penetrating the skin of the subject. In this case the device 100 comprises a securing means, which in this case comprises a self-adhesive patch 101. The micro-needle element 2 may have been applied to the skin of the subject by any suitable means, for example, by using any of the applicators of the apparatus 40, 50, 60 and 70, or directly by hand. The self-adhesive patch 101 comprises a detecting means comprising a pair of measuring electrodes 102, which are similar to the measuring electrodes 34 of the apparatus 1, and which extend downwardly from and are secured to a self-adhesive side 103 of the patch 101. The measuring electrodes 102 are provided for detecting disengagement of the micro-needle element 2 from the skin of the subject. Electrically conductive tracks 104 extend from the measuring electrodes 102 along the self-adhesive side 103 of the patch 101 and are in electrical communication with electrically conductive pads (not shown) on the micro-needle element 2 between the micro-needle element 2 and the self-adhesive patch 101. In this embodiment of the invention the programmable electronic circuit in the micro-needle element 2 is programmed to read the impedance between the measuring electrodes 102 and to compare the impedance between the measuring electrodes 102 with a reference impedance. The reference impedance is of impedance value similar to the impedance which should be detected between the measuring electrodes 102 when penetrating the skin of a subject. The programmable electronic circuit of the micro-needle element 2 is programmed so that on detecting the impedance between the measuring electrodes 102 exceeding the reference impedance, and thus indicating disengagement of the measuring electrodes 102 from the skin of the subject and in turn disengagement of the micro-needles 5 from the skin of the subject, the programmable electronic circuit disables discharge of the medicaments from the micro-needle element 2.

The measuring 102 are of length and are disposed on the patch relative to the micro-needle element in order to remain in penetrating engagement with the skin of the subject for so long as the micro-needles 5 remain in penetrating engagement with the skin of the subject, and to disengage the skin of the subject on disengagement from the skin of the subject of the micro-needles 5 of the micro-needle element 2.

Although not illustrated, an indicating means, such as a light emitting diode may be provided which would be operated under the control of the programmable electronic circuit to indicate disengagement of the micro-needles 5 from the skin of the subject.

It is also envisaged that the measuring electrodes 102 may be adapted to disengage the skin of the subject on the micro-needles 5 of the micro-needle element 2 failing to penetrate the skin of the subject to the desired depth. In which case, on the impedance between the measuring electrodes 102 being indicative of disengagement from the skin of the subject by the measuring electrodes 102, discharge of the medicaments from the micro-needle element 2 would be disabled. Additionally, if a light emitting diode or other such indicating means was provided, the light emitting diode or other indicating means would be activated to indicate that the micro-needles 5 of the micro-needle element 2 were no longer penetrating the skin to the desired depth, and discharge of the medicament or medicaments from the micro-needle element 2 was disabled.

While in the embodiment of the invention described with reference to FIG. 12 the securing means has been described as being a self-adhesive patch, any other suitable securing means may be provided, for example, a strap.

It is also envisaged that the device 100 described with reference to FIG. 12 may also include the micro-needle element 2, and in which case, the micro-needle element and the patch would be simultaneously applied to the skin of the subject and secured thereto by the self-adhesive patch 101.

While the means for determining when the micro-needles of the micro-needle element penetrate the skin of the subject to a desired depth has been described as being provided by a pressure sensor or an impedance detecting means, any other suitable detecting means may be provided.

It is also envisaged that the impedance detecting means may be used for detecting the administering of a medicament to a subject, since it is envisaged that the impedance beneath the skin of the subject adjacent the site where a medicament is administered to the subject would change on administration of the medicament.

While the micro-needle device 3 which has been described with reference to FIGS. 1 to 4 has been described for applying to an arm of a subject, it will be readily apparent to those skilled in the art that the micro-needle device 3 may be attached to any part of the subject by a strap of the appropriate length. For example, the micro-needle device may be attached to a thigh or any part of a leg of the subject by a strap 14 of appropriate length, or to any other limb or part of the subject which would lend itself to securing the micro-needle element thereto with a strap.

Needless to say, the micro-needle device 80 may also be secured by the strap 14 around an arm, leg or any other part of a subject, including the trunk of the body of the subject. Needless to say, where the micro-needle elements 2 are secured to the skin of the subject by a self-adhesive patch, the micro-needle elements 2 may be secured by the self-adhesive patch to any desired or suitable site on the body of a subject, whether on a limb or the trunk of the subject or any other part of the subject.

While the indicating means have been described as producing visually perceptible signals, which in the embodiments of the invention described have been described as being produced by first, second and third light emitting diodes, it will be readily apparent to those skilled in the art that any other suitable indicating means may be provided. Where the visually perceptible signals are produced by indicating means, indicating means other than light emitting diodes may be provided, and indeed, the numbers of light emitting diodes may be different to that described.

It is also envisaged that instead of providing the human sensory perceptible signals as being visually perceptible signals, any other suitable human sensory perceptible signals may be produced, for example, aurally perceptible indicating means, such as an audible alarm, an audible buzzer, an audible bleeper or the like may be provided, and where the indicating means is provided as a bleeper, the rate at which the bleeps are produced by the bleeper or the frequency of the bleeps may vary depending on the pressure which is being applied to the micro-needle element by the strap or by the applicator. For example, initially the rate at which the bleeps would be outputted or the frequency of the bleeps would be relatively low, and as the pressure applied by the strap or the applicator to the micro-needle element increased, the rate at which the bleeps would be produced or their frequency would increase, until the pressure applied by the strap or the applicator to the micro-needle element reached the pressure required to penetrate the micro-needles of the micro-needle element through the skin to the desired depth had been reached, at which stage the bleeper would output a continuous bleep.

It is also envisaged that the human sensory perceptible indicating means may be provided by a vibrator, the frequency of vibration of which would increase as the pressure applied by the strap or the applicator to the micro-needle element increased. Indeed, it will be readily apparent to those skilled in the art that the indicating means may be provided by a combination of different types of indicating means which would produce different types of human sensory perceptible signals. For example, a combination of different indicating means could be provided which would produce a combination of visually perceptible and aurally perceptible signals and indeed even vibratory indicating means could be provided, or any combination of these means.

It is also envisaged that the human sensory perceptible signals may be provided by a display on a visual display screen, where the actual pressure with which the micro-needle device is being urged into engagement with the skin is displayed. The pressure may be displayed numerically or alphanumerically or graphically. Where the pressure is displayed graphically, marks would be provided to indicate the different stages which the pressure crosses. In other words, the first stage mark would give an indication that the pressure with which the micro-needle device is being urged into engagement with the skin of the subject is at a pressure which is approaching the pressure which is sufficient for urging the micro-needles to penetrate the skin of the subject. A second stage mark would indicate that the pressure being applied to the micro-needle element is sufficient for urging the micro-needle element into engagement with the skin of the subject in order that the micro-needles penetrate the skin to the desired depth. A third stage mark would indicate the pressure which is sufficient for maintaining the micro-needles of the micro-needle element in penetrating engagement with the skin of the subject at the desired depth. Thus, as the pressure with which the micro-needle element is being urged into engagement with the skin of the subject increased or decreased, the graphical indication of the pressure would be illustrated the pressure passing the relevant stage marks on the visual display. Such a visual display could be a visual display screen. Alternatively, the indicating means could be provided by a liquid crystal display which would display messages indicating the pressure with which the micro-needle element is being urged into engagement with the skin of the subject. It is also envisaged that the indicating means may be provided by a mechanical display, such as a needle and scale arrangement.

It is also envisaged that the electronic control circuit in the housing 26 of any of the apparatus may be adapted to solely rely on the impedance values detected between the measuring electrodes for determining when the micro-needle element is being urged into engagement with the skin of the subject such that the micro-needles penetrate the skin of the subject to the desired depth, and the impedance between the measuring electrodes could also be used to indicate that the micro-needles are being maintained penetrating the skin of the subject to the desired depth.

Needless to say, it will be appreciated that it may be sufficient to provide an indicating means which produced only one output signal, which would be indicative of the pressure applied by the strap or the applicator to the micro-needle element being sufficient to penetrate the micro-needles through the skin of the subject to the desired depth.

Needless to say, it is envisaged that any combination of events could be indicated by an indicating means.

While the micro-needle elements have been described as comprising a plurality of micro-needles, it is envisaged that in certain cases, the micro-needle element may include only one micro-needle.

While the measuring electrodes have been described as being of length of approximately 0.2 mm, the measuring electrodes may be of any suitable length, and may be of length in the range of 0.1 mm to 1 mm. It is also envisaged that the micro-needles may be of any other suitable length besides 0.2 mm, and it is envisaged that the micro-needles may be of length in the range 0.1 mm to 1 mm. It will also be appreciated that it is not essential that the measuring electrodes be of similar length to that of the micro-needle.

While a particular construction of micro-needle element has been described, it will be readily apparent to those skilled in the art that the apparatus according to the invention may be used in conjunction with any other construction of micro-needle element.

Where it is desired that the measuring electrodes should penetrate the skin prior to penetration of the skin by the micro-needles, the length of the measuring electrodes and their location relative to the micro-needle element will be appropriately arranged, and depending on the location of the measuring electrodes relative to the micro-needle element, the measuring electrodes may be longer or shorter than the micro-needles of the micro-element, however, the measuring electrodes would be of length and disposed relative to the micro-needles of the micro-needle element such that penetration of the skin of the subject by the measuring electrodes would commence prior to penetration of the skin of the subject by the micro-needles.

It is also envisaged that the measuring electrodes may be of length and disposed relative to the micro-needle element so that the measuring electrodes would disengage the skin of the subject prior to disengagement of the skin of the subject by the micro-needles. Such an arrangement would be particularly advantageous where impedance measurement is used for determining when the micro-needles are no longer penetrating the skin of the subject to the desired depth.

In the embodiment of the invention described with reference to FIGS. 10 and 11 it is envisaged that the housing 26 could be omitted and the pressure sensor would be located between the micro-needle element 2 and the self-adhesive patch. The measuring electrodes could be mounted on the micro-needle element or on the self-adhesive patch. The electronic control circuit could be housed in the micro-needle element or the programmable electronic circuit in the micro-needle element could be programmed to carry out the functions carried out by the electronic control circuit.

In the embodiment of the invention described with reference to FIG. 12 it is envisaged that the measuring electrodes may be adapted to abut the skin and not to penetrate the skin. In which case it is envisaged that the measuring electrodes would be provided as disc type electrodes.

It is also envisaged that in the embodiment of the invention described with reference to FIG. 12 the detecting means instead of being provided by an impedance detecting means could be provided by any other suitable detecting means, for example, a pressure sensing means, and in which case the pressure sensing means would typically be located between the micro-needle element and the self-adhesive patch.

1-189. (canceled)

190. Apparatus for applying a micro-needle element, which comprises at least one micro-needle, to a site on the skin of a subject, the apparatus comprising a means for urging the micro-needle element into engagement with the skin of the subject with the at least one micro-needle thereof penetrating the skin, a means for determining when the at least one micro-needle of the micro-needle element penetrates the skin to a desired depth, and an indicating means responsive to the determining means determining that the at least one micro-needle of the micro-needle element has penetrated the skin to the desired depth for producing a first human sensory perceptible signal.

191. Apparatus as claimed in claim 190 in which the determining means for determining when the at least one micro-needle penetrates the skin of the subject to a desired depth comprises a pair of measuring electrodes, the measuring electrodes being urgeable by the urging means into penetrating engagement with the skin of the subject with the at least one micro-needle, and an electrical impedance detecting means for detecting electrical impedance beneath the skin of the subject between the measuring electrodes, and preferably, the measuring electrodes are located spaced apart from each other, and advantageously, the measuring electrodes are located so that the depth to which the measuring electrodes penetrate beneath the skin of the subject in response to the urging means urging the micro-needle element into engagement with the skin of the subject is indicative of the depth of penetration of the at least one micro-needle into the skin of the subject, and preferably, the measuring electrodes are located on one of the urging means and the micro-needle element.

192. Apparatus as claimed in claim 191 in which each measuring electrode comprises a pointed element, and preferably, each measuring electrode comprises a micro-needle, and advantageously, each measuring electrode is of an electrically conductive material, and preferably, each measuring electrode is of length substantially similar to the length of the at least one micro-needle, and alternatively,
each measuring electrode is of length relative to the length of the at least one micro-needle so that the measuring electrodes penetrate the skin of the subject just prior to penetration of the skin of the subject by the at least one micro-needle, and advantageously, each measuring electrode is of length at least 10% longer than the at least one micro-needle, and preferably, in the range of 20% to 35% longer than the at least one micro-needle, and advantageously, in the range of 30% to 33% longer than the at least one micro-needle, and preferably, the impedance detecting means is located on one of the urging means and the micro-needle element.

193. Apparatus as claimed in claim 190 in which the determining means for determining when the at least one micro-needle penetrates the skin of the subject to the desired depth comprises a pressure sensing means for detecting the pressure under which the micro-needle element is urged into engagement with the skin of the subject, and preferably, the pressure sensing means comprises one of a piezoelectric crystal and a strain gauge, and advantageously, the pressure sensing means is located on one of the urging means and the micro-needle element, and preferably, the pressure sensing means is located between the urging means and the micro-needle element, and advantageously, the indicating means is responsive to the pressure sensing means detecting that the pressure with which the micro-needle element is urged into engagement with the skin of the subject is sufficient for urging the at least one micro-needle of the micro-needle element to penetrate the skin of the subject to the desired depth for producing the first human sensory perceptible signal, and preferably, the indicating means is responsive to the pressure sensing means detecting when the pressure with which the micro-needle element is urged into engagement with the skin of the subject is sufficient for maintaining the at least one micro-needle penetrating the skin of the subject to the desired depth for producing a second human sensory perceptible signal, and advantageously, the indicating means is responsive to the pressure sensing means detecting that the pressure with which the micro-needle element is being urged into engagement with the skin of the subject is approaching the pressure which should be sufficient to urge the at least one micro-needle to penetrate the skin to the desired depth for producing a third human sensory perceptible signal, and preferably, the indicating means is responsive to the pressure sensing means detecting that the pressure with which the micro-needle element is being urged into engagement with the skin of the subject is insufficient for penetrating the skin of the subject by the at least one micro-needle for producing a fourth human sensory perceptible signal.

194. Apparatus as claimed in claim 190 in which the indicating means comprises a visual display means, and preferably, each human sensory perceptible signal produced by the indicating means is one of a visually perceptible signal and an aurally perceptible signal, and advantageously, each human sensory perceptible signal produced by the indicating means is a visually perceptible signal, and preferably, the first human sensory perceptible signal is provided by activating a light of a first colour, and advantageously, the second human sensory perceptible signal is provided by activating a light of a second colour, and preferably, the third human sensory perceptible signal is provided by activating the light of one of the first and second colours to flash or by activating a light of a different colour to that of the first and second colours, and advantageously, the fourth human sensory perceptible signal is provided by activating a light of a different colour to that of the first and second colours, and preferably, each light is provided by a light emitting diode, and advantageously, each light is located on one of the urging means and the micro-needle element.

195. Apparatus as claimed in claim 190 in which a monitoring means is provided for detecting disengagement of the micro-needle element from the skin of the subject, and preferably, the indicating means is responsive to the monitoring means detecting disengagement of the micro-needle element from the skin of the subject for producing a fifth human sensory perceptible signal indicating disengagement of the micro-needle device, and advantageously, the fifth human sensory perceptible signal is one of a visually and an aurally perceptible signal.

196. Apparatus as claimed in claim 195 in which the monitoring means is responsive to disengagement of the micro-needle element from the skin of the subject for disabling a medicament delivery means of the micro-needle element, and prererably, the means for determining when the at least one micro-needle of the micro-needle element has penetrated the skin of the subject to the desired depth is adapted for detecting disengagement of the micro-needle element from the skin of the subject, and preferably, the monitoring means is responsive to the determining means determining disengagement of the micro-needle element from the skin of the subject, and advantageously, the monitoring means is responsive to the pressure sensing means detecting pressure indicative of disengagement of the micro-needle element from the skin of the subject, and preferably, the monitoring means is responsive to the electrical impedance detecting means detecting electrical impedance between the measuring electrodes being indicative of disengagement of the micro-needle element from the skin of the subject, and advantageously, the urging means is adapted for retaining the micro-needle element on the skin of the subject with the at least one micro-needle penetrating through the skin of the subject, and preferably, the urging means is adapted for carrying the micro-needle element between the urging means and the skin of the subject, and advantageously, the urging means comprises one of a strap and a patch, and preferably, the one of the strap and the patch is adapted for carrying the micro-needle element between the one of the strap and the patch and the skin of the subject, and preferably, the patch comprises a means for securing the patch to the skin of the subject, and advantageously, the patch comprises a self-adhesive coating adapted for securing the patch to the skin of the subject, and preferably, the strap is adapted for extending around one of a limb or a trunk of the subject, and advantageously, an adjusting means is provided for adjusting tension in the strap for varying the pressure with which the micro-needle element is urged into engagement with the skin of the subject, and preferably, the adjusting means comprises an adjustable clasp for adjustably securing the strap to the subject.

197. Apparatus as claimed in claim 196 in which an electrical power supply means is located on one of the micro-needle element and the strap, and an electrically conducting means extends between the micro-needle element and the strap for conducting electrical power from the power supply means from the one of the micro-needle element and the strap, to the other of the micro-needle element and the strap, and preferably, the determining means for determining when the at least one micro-needle of the micro-needle element penetrates the skin of the subject to the desired depth is located on the strap, and the determining means is powered by the electrical power supply means through the electrical conducting means, and advantageously, the electrical power supply means is located on the micro-needle element and the determining means is powered by the electrical power supply means through the electrical conducting means, and alternatively, the electrical power supply means is located on the strap, and the micro-needle element is powered by the electrical power supply means through the electrical conducting means, and preferably, the determining means comprises the pressure sensing means and the electrical impedance detecting means.

198. Apparatus as claimed in claim 190 in which the apparatus comprises the micro-needle element, and preferably, the micro-needle element comprises a plurality of micro-needles, and advantageously, the micro-needle element is programmable for sequentially dispensing doses of one or more medicaments therefrom, and preferably, a means for recording each dose of the one or more medicaments and the time of administering of the dose is provided for subsequent analysis, and advantageously, the means for recording each dose and the time of administering the dose is provided on one of the urging means and the micro-needle element, and preferably, the means for recording each dose and the time of administering the dose is provided on the micro-needle element, and advantageously, the apparatus comprises an applicator for applying the micro-needle element to the skin of the subject, the applicator comprising a housing, and the urging means is located in the housing, and preferably, the determining means for determining when the at least one micro-needle of the micro-needle element penetrates the skin of the subject to the desired depth is located on the applicator, and advantageously, the urging means comprises a piston slideable within the applicator, and preferably, the piston is powered by one of a pneumatic powering means, an electrical powering means and a spring urging means, and advantageously, the indicating means is provided on the applicator.

199. A micro-needle device comprising a micro-needle element having at least one micro-needle, an urging means for urging the micro-needle element into engagement with the skin of a subject and for retaining the micro-needle element in engagement with the skin of the subject with the at least one micro-needle penetrating the skin of the subject, a means for determining when the at least one micro-needle has penetrated the skin of the subject to a desired depth, and an indicating means responsive to the determining means for indicating when the at least one micro-needle has penetrated the skin of the subject to the desired depth.

200. An applicator for applying a micro-needle element comprising at least one micro-needle to the skin of a subject, the applicator comprising a housing defining an abutment surface for abutting the micro-needle element, an urging means located in the housing for engaging the micro-needle element, the urging means being adapted to impart a pressure to the micro-needle element sufficient for urging the micro-needle element into engagement with the skin of the subject with the at least one micro-needle penetrating the skin of the subject to a desired depth when the micro-needle element is abutting the abutment surface.

201. An applicator as claimed in claim 200 in which the urging means acts through the abutment surface, and preferably, the urging means comprises a spring, and preferably, the urging means comprises a compression spring, and advantageously, the urging means is adapted to act between the housing and the micro-needle element, and preferably, a bore extends into the housing from the abutment surface for accommodating the urging means therein.

202. A method for applying a micro-needle element having at least one micro-needle to a site on the skin of a subject, the method comprising urging the micro-needle element into engagement with the skin of the subject with the at least one micro-needle thereof penetrating the skin, determining when the at least one micro-needle penetrates the skin to a desired depth, and producing a first human sensory perceptible signal when the at least one micro-needle has penetrated the skin of the subject to the desired depth.

203. A method as claimed in claim 202 in which determining when the at least one micro-needle penetrates the skin to a desired depth is carried out by one of determining the electrical impedance between a pair of measuring electrodes adapted to penetrate the skin of the subject with the at least one micro-needle, and sensing the pressure with which the micro-needle element is urged into engagement with the skin of the subject, and preferably, the first human sensory perceptible signal is produced when the pressure with which the micro-needle element is being urged into engagement with the skin of the subject is sufficient to urge the at least one micro-needle of the micro-needle element to penetrate the skin of the subject to the desired depth, and preferably, a second human sensory perceptible signal is produced when the pressure with which the micro-needle element is being urged into engagement with the skin of the subject is sufficient for maintaining the at least one micro-needle penetrating the skin of the subject to the desired depth, and advantageously, a third human sensory perceptible signal is produced when the pressure with which the micro-needle element is being urged into engagement with the skin of the subject is approaching the pressure which should be sufficient to urge the at least one micro-needle to penetrate the skin to the desired depth, and preferably, a fourth human sensory perceptible signal is produced when the pressure with which the micro-needle element is being urged into engagement with the skin of the subject is insufficient to urge the at least one micro-needle to penetrate the skin of the subject, and preferably, each human sensory perceptible signal is displayed on a visual display means, and advantageously, each human sensory perceptible signal is produced as one of a visually perceptible signal and an aurally perceptible signal.

204. A method as claimed in claim 202 in which disengagement of the micro-needle element from the skin of the subject is detected, preferably, disengagement of the micro-needle element from the skin of the subject is detected by one of detecting the electrical impedance between the measuring electrodes and detecting the pressure with which the micro-needle element is being urged into engagement with the skin of the subject, and advantageously, delivery of a medicament from the micro-needle element is disabled in response to detection of the micro-needle element disengaging the skin of the subject.

205. A method as claimed in claim 202 in which the micro-needle element is urged into engagement with the skin of the subject by one of a strap, an applicator and a patch, and preferably, the strap is adapted to extend around a limb or trunk of the subject, and advantageously, the patch is a self-adhesive patch.

206. A device for retaining a micro-needle element in engagement with the skin of a subject with micro-needles of the micro-needle element penetrating the skin of the subject, the device comprising a securing means for securing the micro-needle element to the skin of the subject, and a detecting means for detecting disengagement of the micro-needle element from the skin of the subject.

207. A device as claimed in claim 206 in which the detecting means comprises a pair of spaced apart measuring electrodes adapted to penetrate the skin of the subject, and a means for determining the impedance between the measuring electrodes, the measuring electrodes being adapted to remain in penetrating engagement with the skin of the subject while the micro-needles remain in penetrating engagement with the skin of the subject, and preferably, a means is provided for comparing the impedance between the measuring electrodes with a reference impedance to detect disengagement of the micro-needle element from the skin of the subject, and advantageously, the measuring electrodes are located on the securing means adjacent the micro-needle element, and preferably, the detecting means is adapted to output a disabling signal for disabling discharge of a medicament from the micro-needle device in response to detection of disengagement of the micro-needle element from the subject, and advantageously, the securing means comprises one of a self-adhesive patch and a strap, and preferably, the device comprises a micro-needle element, and the micro-needle element is responsive to the disabling signal for disabling discharge of a medicament from the micro-needle element, and preferably, each measuring electrode comprises a pointed element, and advantageously, each measuring electrode comprises a micro-needle, and preferably, each measuring electrode is of an electrically conductive material.

208. A method for retaining a micro-needle element in engagement with the skin of a subject with micro-needles of the micro-needle element penetrating the skin of the subject, the method comprising securing the micro-needle element to the skin of the subject with a securing means, and detecting disengagement of the micro-needle element from the skin of the subject by a detecting means.

209. A method as claimed in claim 208 in which the detecting means comprises a pair of spaced apart measuring electrodes adapted to penetrate the skin of the subject, and a means for determining the impedance between the measuring electrodes, the measuring electrodes being adapted to remain in penetrating engagement with the skin of the subject while the micro-needles remain in penetrating engagement with the skin of the subject, and preferably, a means is provided for comparing the impedance between the measuring electrodes with a reference impedance to detect disengagement of the micro-needle element from the skin of the subject, and advantageously, in which the measuring electrodes are located on the securing means adjacent the micro-needle element, and preferably, the detecting means is adapted to output a disabling signal for disabling discharge of a medicament from the micro-needle device in response to detection of disengagement of the micro-needle element from the subject, and advantageously, the securing means comprises one of a self-adhesive patch and a strap, and preferably, the micro-needle element is attached to the securing means, and the micro-needle element is responsive to the disabling signal for disabling discharge of a medicament from the micro-needle element.
